(19) United States
(12) Patent Application Publication
(10) Pub. No.: US 2022/0061673 A1
(43) Pub. Date: Mar. 3, 2022

(54) Device and Method for Tissue Analysis

(71) Applicant: Erbe Elektromedizin GmbH, Tuebingen (DE)

(72) Inventors: Jan Jaeger, Tuebingen (DE); Yannick Daniel, Balingen (DE)

(21) Appl. No.: 17/408,791

(22) Filed: Aug. 23, 2021

(30) Foreign Application Priority Data
Sep. 3, 2020 (EP) 20194405.5

(51) Int. Cl. A61B 5/00; A61B 90/30

(52) U.S. Cl. CPC A61B 5/0075; A61B 90/30; A61B 2505/05; A61B 5/7264; A61B 2562/0238; A61B 5/0062

(57) ABSTRACT

A tissue analysis device is described having a light receiving device and a spectrometer device for determination of tissue characteristics which includes an evaluation device connected with an assignment device. The evaluation device serves for determination of at least one tissue characteristic of a biological tissue, e.g. of its type or an infection with a disease. The assignment device serves for assignment of a suitable transmission curve model that models the contamination of the light receiving device. For different degrees of contamination different transmission curve models are provided that comprise reliability values for each tissue characteristic that can be determined respectively. Not only the tissue analysis can be achieved, but also the indication of the reliability with which the analysis has been carried out, i.e. how reliable the indication of the tissue characteristic is.

### RELATED APPLICATION(S)

This application claims the benefit of European Patent Application No. 20194405.5, filed Sep. 3, 2020, the contents of which are incorporated herein by reference as if fully rewritten herein.

### TECHNICAL FIELD

The invention refers to a device as well as a method for tissue analysis, particularly for integration in a surgical device.

### BACKGROUND

It is known to influence biological tissue by means of electrical sparks or an electrically created plasma and to analyze the light created thereby in order to draw conclusions on the treated tissue. For this WO 2011/055369 A2 discloses a catheter for plasma ablation, wherein the catheter comprises a light receiving device in the form of an optical fiber that is arranged in proximity to an ablation electrode. The light received from the optical fiber is supplied to an analysis device, e.g. a spectrometer, and is subject to a spectral analysis to be able to particularly distinguish whether the electrical spark acts upon depositions, so-called plaques, particularly the phosphorous line of the spectrum is monitored (254 nm).

The determination of the kind of treated tissue or other tissue characteristics, as for example non-malignant or malignant, is also known from EP 2 659 846 B1. For tissue recognition there the light of a spark acting upon biological tissue is analyzed in terms of its spectral composition. Also here the receipt of light originating from the spark in the proximity of the spark is necessary.

It is known that during the execution of interventions on living tissue by means of sparks, depositions can be formed on light receiving windows that affect the tissue analysis. As remedy, EP 2 815 713 B1 proposes to form the light receiving window by a resting or flowing liquid body. This shall counteract a tendency for depositions of carbon black or other contaminations on the light receiving window. However, liquid bodies have no geometrically defined shape and they cannot be used particularly in plasma applications that are accompanied by excessive heat development.

From these and other influencing factors a certain situation-dependent uncertainty results during the optical determination of tissue characteristics based on the light emitted from a spark or plasma.

It is the object of the invention to provide a concept for determination of the reliability of the tissue recognition.

### SUMMARY

This object is solved by means of a device and also by means of a method as disclosed herein.

The tissue analysis device according to one form of the invention serves for tissue recognition. The spectral composition of the light is evaluated for tissue recognition that is created by the influence of a spark on biological tissue. For this the tissue analysis device comprises a light receiving device for receiving light that is created due to the influence of an electrical spark or plasma on biological tissue. The light receiving device can be, for example, the end surface of an optical fiber, an objective lens attached there or the like. Particularly the light receiving device is preferably arranged in the proximity of an electrode of a respective surgical instrument and thus close to the forming spark or plasma. For this reason the light receiving device can be subject to a certain contamination or also degradation. The contamination can result from depositions of carbon black, tissue particles, dust, salt crystals or similar. The light received from the light receiving device is supplied to a spectrometer device that determines the light intensities at least at one, preferably at multiple wavelengths of the light and provides signals to an evaluation device characterizing the light intensities. The evaluation device determines data from the signals characterizing the light intensities, wherein the data characterize the at least one tissue characteristic. Tissue characteristic means any feature characterizing the tissue, such as for example the tissue type (bones, blood, connected tissue, muscle, nerves, organ tissue, etc. or also different tumor tissues). A tissue characteristic to be distinguished can also be a characteristic within a tissue type, for example, whether it is healthy or sore tissue, tumor tissue, infected tissue, dead tissue or the like.

An assignment device is part of the tissue analysis device for assignment of a reliability value to the data determined by the evaluation device. The assignment device is configured to determine the reliability value based on the contamination of the light receiving device. The determination of the contamination is carried out indirectly in that the spectrum is evaluated that is created by the spectrometer device. The spectrum can originate from a light source of known spectral composition or also from light that a spark emits that acts upon tissue. If the light originates from a light source of known spectral composition, the evaluation is particularly simple. From the spectrum provided by the spectrometer device it can then be directly concluded on the type and degree of contamination and thus the contamination can be classified. Different contamination classes can correspond to different transmission curves that, like filter curves, influence the light emitted by the light source and received by the light receiving device. Reliability values for different tissue characteristics, for example different tissue types, can be assigned to different transmission curves. For example, also in case of intense contamination, certain tissue types can still be recognized in a quite reliable manner, whereas other tissue types cannot be recognized in a quite reliable manner, even in case of low contamination.

However, in this approach for contamination determination, a test device has to be used from time to time in order to provide light of known spectral composition to the light receiving device. A light source may be part of the test device that is arranged such that light emitted therefrom is detected by the light receiving device. The light source can be, for example, a light source that is always illuminated, if no spark is present at the electrode of a respective instrument. If a spark is present, it can be switched off (non-illuminated).

As an alternative, the test device can use a surgical area illumination as light source. This particularly applies, if the surgical area illumination emits light in the wavelength range that is relevant for the tissue recognition. In addition, it is advantageous, if the brightness of the surgical area illumination is sufficiently constant. It is possible to carry out the test, if and—as an option—always if the instrument is just not activated.

In another embodiment such a light source is omitted or the surgical area illumination is not used for test purposes. Rather the light emitted from the spark and received by the light receiving device is supplied to the spectrometer device that determines the assigned spectrum. In this case the assignment device can be connected with a data block that contains multiple transmission curve models that are characteristic for different contaminations. The transmission curve models are in turn filter curves that can distinguish in their qualitative shape and in their wavelength dependent attenuation values. In this case, the assignment device is configured to identify the transmission curve model matching with the recorded spectrum.

In both embodiments a data set is assigned to the transmission curve model that assigns different reliability values to different tissue characteristics. These data are used for the further evaluation of the spectrum. If the manufacturer or user is, for example, defining a reliability value of at least 98% and if the valid transmission curve model in the example comprises a reliability above this limit only for some of the tissue characteristics that can be determined in principle, the assignment device can be configured to only indicate those tissue types for which a sufficient reliability is provided. As an alternative, the correlated reliability values can be indicated with each determined tissue characteristic. Low reliabilities can be signalized optically or acoustically in order to avoid treatment errors.

### BRIEF DESCRIPTION OF THE DRAWINGS

Further details of advantageous embodiments of the invention are derived from the dependent claims, from the figures, the drawings or from the respective description. The drawings show:

FIG. 1 a tissue analysis device in a schematic block diagram illustration,

FIG. 2 a part of the tissue analysis device for clarification of the basic operating principle,

FIG. 3 transmission curve models for different contaminations of the light receiving device,

FIG. 4 the spatial illustration of a reliability value dependent from the contamination type and magnitude (degree) of contamination,

FIG. 5 different reliability curves with increasing contamination for different tissue characteristics,

FIG. 6 a tissue analysis device with test device as block diagram.

### DETAILED DESCRIPTION

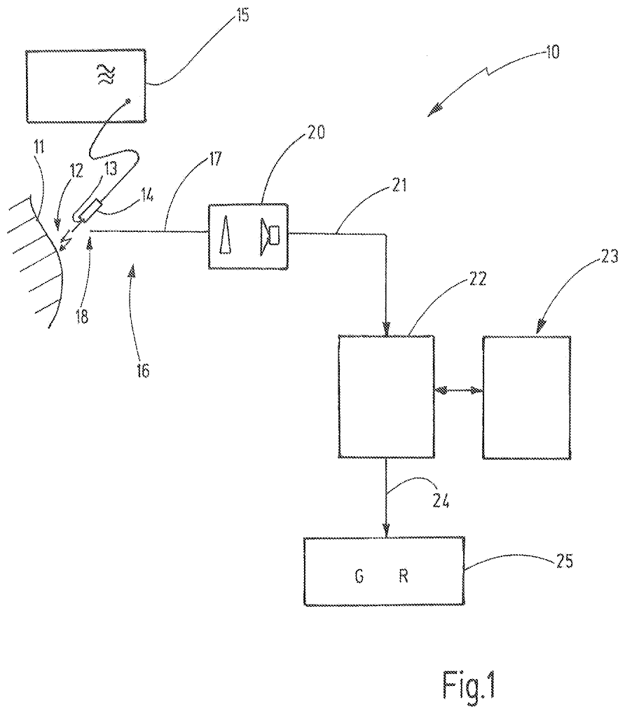

FIG. 1 illustrates a tissue analysis device 10 that is configured to recognize particular tissue characteristics G based on light that results from a spark 12 influencing biological tissue 11. The spark 12 can originate from an electrode 13 of a surgical instrument 14 that is supplied with electrical power from an apparatus 15. For example, the electrode 13 is in this way supplied with radio frequency current that is flowing via the tissue 11 and a respective counter electrode. For example, the instrument 14 is a monopolar instrument that requires a neutral electrode that has to be attached to the patient—and that is not illustrated in FIG. 1—in order to close the electrical circuit. The tissue analysis device 10 can also cooperate with a bipolar instrument that comprises two or more electrodes between which the spark is lighted. As soon as the spark 12 or a plasma jet created by it touches the tissue 11, a light appearance is created, the spectrum of which allows conclusions about the type and condition, i.e. characteristics of tissue 11.

The tissue analysis device 10 that can be part of the instrument 14 or can also be configured as separate unit, serves for determination of such tissue characteristics G. The tissue analysis device 10 is configured to determine and indicate a relevant characteristic of tissue, e.g. what type of tissue it is that is in contact with the spark (e.g. connective tissue or organ tissue).

A light receiving device 16, e.g. in the form of a light conductor 17, the distal end 18 of which forms a light receiving window and is arranged in the proximity of the electrode 13 and/or the spark 12, is part of the tissue analysis device 10. The light receiving window can also be formed by a lens, an objective or the like.

The light receiving device 16 is connected to a spectrometer device 20 and supplies the received light resulting from the spark 12 to the spectrometer device 20. The spectrometer device 20 is configured to determine the spectrum of the light. The spectrum is characterized by the light intensities that are present at different wavelengths of the light. Any kind of spectrometer is suitable as spectrometer device 20 that is suitable to output signals on a conductor 21 that characterize the different light intensities at different light wavelengths.

The conductor 21 connects the spectrometer device 20 with an evaluation device 22 that is configured to determine tissue characteristics G from the spectra measured by a spectrometer device 20 (i.e. from the signals output therefrom). The tissue characteristics G to be determined can be the tissue type or also specific features of a tissue type. For example, a tissue type (muscle tissue, bone tissue, fat tissue, blood, etc.) can be examined for particular features (ion content, phosphorous content or other subtle features). For this the evaluation device can be trained based on numerous different tissue samples and can comprise respective learning algorithms or other learning structures. For this the evaluation device 22 can also use explicitly defined calculation algorithms or other evaluation algorithms. The evaluation device 22 creates data D that characterize characteristics of the tissue. For example, data D can be appropriate to indicate the tissue type, to distinguish malign from non-malign tissue or the like.

The tissue analysis device 10 comprises in addition an assignment device 23 that is configured to assign reliability values R to data D. Data D as well as reliability values R can be provided to a display device 25 via a conductor 24. The reliability value is determined based on a transmission measurement and applies for all subsequent data D until the next transmission measurement. Thus, reliability of the tissue classification and potentially also a reduced reliability is assigned quasi in advance to the measurements.

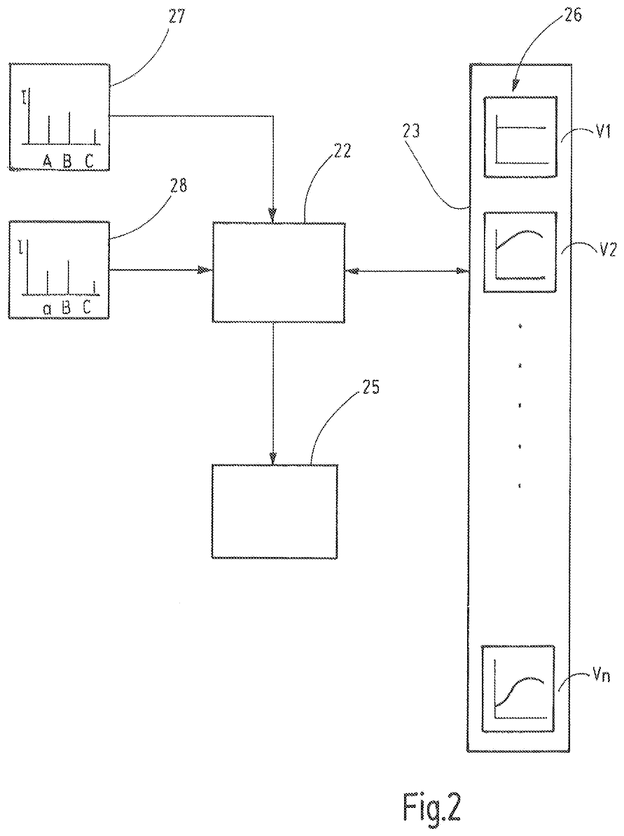

The evaluation device 22, the assignment device 23 connected therewith and their cooperation are apparent in more detail from FIG. 2. The assignment device 23 serves particularly to determine how reliable a specific tissue characteristic G can be determined. Generally speaking, the reliability decreases with increasing contamination of the light receiving device 16. However, this does not apply equally for all tissue characteristics G to be determined. For example, as fat tissue is still well distinguishable from bone tissue, also if the light receiving device or its light receiving window is already subject to a remarkable contamination, for example the more subtle distinguishing of similar tissue types or the distinguishing of non-malign and malign tissue can become unreliable already with less degrees of contamination.

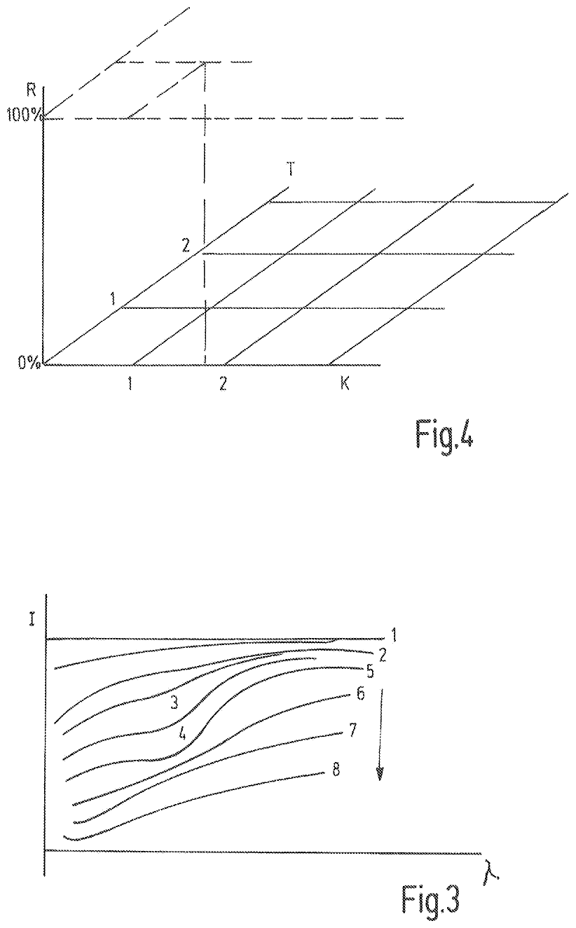

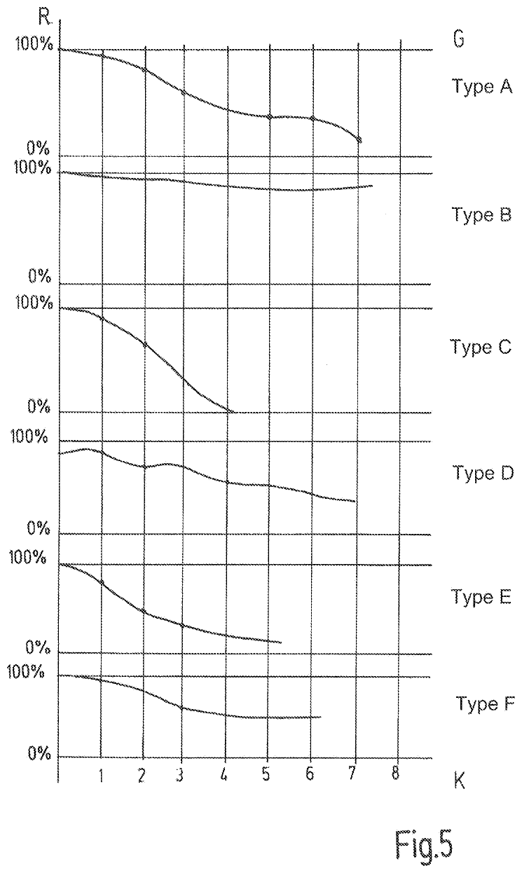

Contamination of a light receiving window changes the transmission characteristics thereof. A deposition on the light receiving window has the effect similar to a filter and thus has a spectrum distorting effect. The assignment device can provide a variety of transmission curve models 26 that are characteristic for different contaminations V (V1, V2 . . . Vn). For example, while the transmission curve for no contamination V1 has an all-pass characteristic, the transmission curves V2 . . . Vn are transmission curves having low-pass or band-pass characteristic or are transmission curves having filter curves with multiple minima, maxima and/or inflection points. For this FIG. 3 illustrates the typical change of the transmission curves during increasing contamination from 1 to 9. The curves show the light intensity I illustrated depending on the light wavelength λ.

Different reliability values R are obtained for each transmission curve model 26 with the different contaminations V1 to Vn during the recognition of tissue characteristics G. This is illustrated for different tissue types of type A to type F in FIG. 5. Again, contaminations are numbered from 1 to 8 similar to FIG. 3, which corresponds to increasing degrees of contamination. In case of contamination degree 1 in nearly all of the tissue types, apart from the types C and E, still a reliability of nearly 100% is obtained. With increasing degrees of contamination (2, 3, 4, etc.) the reliability decreases depending on the tissue type, wherein the reductions have different amounts. This applies for the types A to F of the tissue as well as for other tissue characteristics G or tissue features. With a defined degree of contamination of, for example, 3 some tissue characteristics G, e.g. the characteristic of type B, can still be determined reliably, type D reliable to some extent and other types or characteristics not reliable any more, for example. This information, namely which tissue characteristic G can be determined with which reliability is part of each transmission curve model 26 of V1 to Vn.

The evaluation device 22 first determines the desired tissue characteristic G, e.g. the tissue type. The assignment device 23 then assigns a respective reliability value R based on the respectively valid transmission curve model 26 (V1, V2 . . . or Vn) to this characteristic. Both data can be provided to the display device 25 via conductor 24 and displayed there. Thereby the data D characterize, for example, the identified tissue type or another tissue characteristic G. The reliability value R thereby characterizes the reliability with which the tissue characteristic G has been determined.

The determination of the reliability value R can be carried out prior to the actual application at least in one embodiment of the invention. In doing so, reliability values R can be assigned subsequently also to transmissions measured during operation. For example, spectra can be recorded and the tissue can be classified prior to the application on a test tissue with a fiber having 100% transmission. Subsequently, different transmission curve models can be used in order to simulate different contaminations. With these transmission curve models the tissue can then be classified again. By comparison with the tissue classified at 100% transmission it can be determined which transmission curve model is deteriorated in which degree in terms of the reliability of the tissue analysis.

Then I can use it during the application in order to decide whether a fiber having a specific transmission measured during the application is still good enough for the present tissue classification.

It is also possible to block the indication of tissue characteristics G (data D), if the reliability value R falls below a defined or selected limit.

The transmission curve models 26 can be one-dimensional models that characterize only the increasing contamination, as obvious from FIGS. 3 and 5. It is, however, also possible to configure the classification of the different contaminations, such that the contamination types T and the magnitude or degree of contamination K can be distinguished. For example, the contamination type can depend on the kind of contamination (carbon black deposition, deposition of tissue, deposition of other fumes). This can be particularly the case, if the respective particle size is different. Then the degree of contamination K can characterize the thickness of the formed deposition. For example, the contamination types T can define different filter curves, e.g. low-pass or band-pass or a combination of different basic characteristics, whereas the degree of contamination characterizes cutoff frequencies, slopes or other parameters of the filter curves.

The assignment device 23 has to select a model from the provided transmission curve models 26 that matches the respective contamination best. For this reference is made to FIG. 2. With a first spectrum 27 a spectrum shall be symbolized as recorded in case of a non-contaminated light receiving device 16 on a specific tissue type, e.g. muscle tissue. Multiple spectral lines A, B, C are present that occur with different intensities I. The number of spectral lines and their intensities depend on the respective tissue type. They are only illustrated figuratively in FIG. 2. If now, due to contamination of the light receiving device 16, a different spectrum 28 is recorded on the respective tissue, e.g. muscle tissue, the spectral lines thereof are modified due to the contamination. While the spectrum 27 shows spectral lines A, B, C, the spectrum 28 comprises the spectral lines a, B, C, i.e. one or more spectral lines have a lower intensity I than it would have been the case with a clean light receiving device 16. If however the surgeon knows from which tissue the spectrum results and if this information is available for the evaluation device 22, it can determine based on the modification of the spectrum 28 compared with the ideal spectrum 27, which of the transmission curve models 26 must have affected the modification of the spectrum and can select a respective transmission curve model from the group of the available models V1 to Vn. Upon selection of the transmission curve model the evaluation device 22 concurrently obtains an evaluation of the reliability from the assignment device 23 with which specific tissue characteristics can be recognized. Because the reliability values R are known for all other tissue characteristics G for the transmission curve model, the surgeon can continuously work with his/her instrument and can influence different tissue types while the display device 25 always indicates the determined tissue characteristic G (e.g. the tissue type) and the assigned reliability value R to him/her with which the tissue characteristic G (e.g. tissue type) has been recognized.

The selection of the respective transmission curve models can be checked after predefined time intervals, e.g. after one or more seconds respectively. It is also possible to extrapolate a transmission model based on the activation time and the contamination rate so far. The extrapolation can be checked in defined time intervals or at given opportunities, e.g. between activation of the instrument by means of a transmission measurement. The surgeon does not need to carry out a separate calibration.

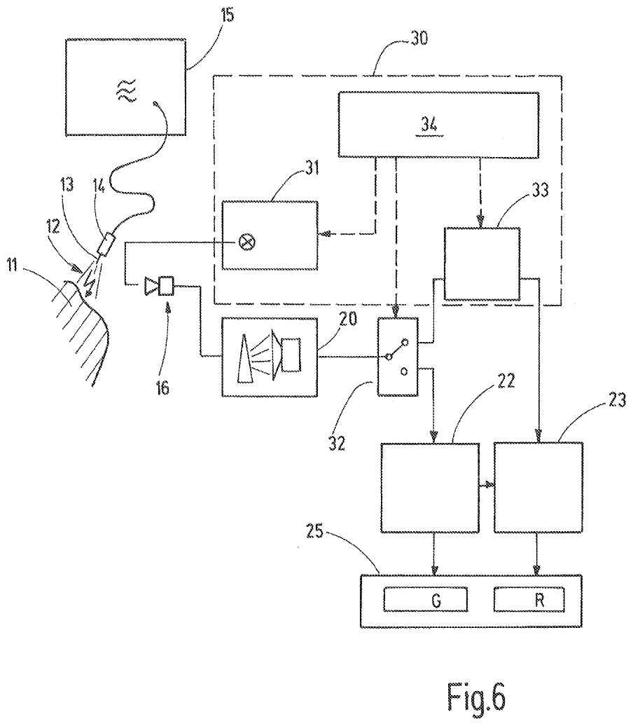

In a modification of the invention it is also possible to provide a test device 30, as illustrated in FIG. 6. It comprises a light source 31 by means of which light with a defined spectral composition can be supplied to the light receiving window of the light receiving device 16. A control device 34 thereby coordinates the test process in that it activates the light source 31 from time to time, such that light enters the light receiving window of the light receiving device 16. The spectrometer 20 outputs its data in this test condition via switch 32 to a transmission classifier 33 that determines the degree of contamination K and/or contamination type T. The initiating of a test and control of the progress thereof is subject to a control device 34 that can be part of the test device 30.

Alternatively, the surgical area illumination can be used as light source 31. For this, short operation breaks can be used during which the electrode 13 does not emit a spark 12. The control device 34 can use these operation breaks and process a routine for determination of the suitable transmission curve model respectively.

The degree of contamination K and/or the contamination type T provided by the transmission classifier 33 is supplied to the assignment device 23 that in turn selects the matching transmission curve model 26 analog to the previous description provided with reference to FIG. 2. The transmission measurement provides information about the degree of contamination. The transmission classifier 33 and/or the assignment device 23 can be configured to determine a comparable transmission curve model (selected from a pool) and to determine which tissue classifications are possible with which reliability R. Thereby the transmission classifier 33 and/or the assignment device 23 can be configured to carry out an interpolation between comparable transmission curve models based on the degree of contamination and/or the contamination type.

If the test is terminated, the control device 34 switches the switch 32 again such that the signals supplied by the spectrometer 20 are directed to the evaluation device 22 that now again determines the desired tissue characteristics from the spectrum gained from the spark light. These tissue characteristics are provided in form of data to the display device 25 that indicates the tissue characteristics.

In doing so, the transmission classifier 33 and the assignment device 23 can make a prediction from the measured transmission how good the result of a tissue classification will be. For example, the tissue classification provides the correct result by 92% by using the contaminated actual fiber. In case of a clean fiber, the tissue classifier provides the correct result, e.g. by 96%. By means of the transmission classifier 33, measured transmissions can now be subdivided in those that achieve the, for example, 92% and better and those for which the expected quality of the tissue classification is below 92%.

Data D are provided to the tissue classifier 33 for tissue determination that determines the type of tissue. The latter and the determined reliability value R are now supplied to the display device 25. It can display the reliability value R. It can also signalize if it goes below a threshold. The threshold can be fixed or defined in a variable manner.

A tissue analysis device 10 according to one form the invention having a light receiving device 16 and a spectrometer device 20 for determination of tissue characteristics G comprises for this purpose an evaluation device 22 that is connected with an assignment device 23. The evaluation device 22 serves for determination of at least one tissue characteristic G of a biological tissue, e.g. of its type or an infection with a disease. The assignment device serves for assignment of a suitable transmission curve model 26 that models the contamination of the light receiving device 16. For different degrees of contamination different transmission curve models are provided that comprise reliability values R for each tissue characteristic G that can be determined respectively. With the inventive concept not only the tissue analysis can be achieved, but in addition also the indication of the reliability with which the analysis has been carried out, i.e. how reliable the indication of the tissue characteristic G is.

### LIST OF REFERENCE SIGNS

10 tissue analysis device
11 biological tissue
12 spark
13 electrode
14 instrument
15 apparatus
16 light receiving device
17 light conductor
18 distal end of light conductor 17
20 spectrometer device
21 conductor
22 evaluation device
G tissue characteristic
D data
23 assignment device
24 conductor
25 display device
26 transmission curve models
V, V1 Vn contamination/transmission curves
I light intensity
λ light wavelength
T contamination type
K degree of contamination
R reliability value
27 first spectrum
28 different spectrum
30 test device
31 light source
32 switch
33 transmission classifier
34 control device
35 fiber coupler

1. A tissue analysis device (10) for integration in a surgical instrument (14) and/or an apparatus (15) for supplying the instrument, the tissue analysis device comprising:
a light receiving device (16) configured to receive light related to an electrical spark (12) or plasma on tissue (11);
a spectrometer (20) for determination of light intensities at different wavelengths of the light;
an evaluation device (22) for determination of data (D) from the light intensities characterizing at least one tissue characteristic (G); and
an assignment device (23) for assignment and/or determination of a reliability value (R) corresponding to the at least one tissue characteristic (G) based on a determined contamination (V).

2. The tissue analysis device according to claim 1, wherein the assignment device (23) is configured to carry out a classification of the contamination (V) based on the light intensities determined by the spectrometer device.

3. The tissue analysis device according to claim 1, further comprising a test device (30) configured to classify the contamination (V).

4. The tissue analysis device according to claim 3, wherein the classification of the contamination (V) comprises the assignment of the contamination (V) to one or more contamination types (T).

5. The tissue analysis device according to claim 3, wherein the test device (30) is configured to detect the degree (K) of the contamination (V).

6. The tissue analysis device according to claim 4, wherein the assignment device (23) is configured to determine the reliability value (R) based on the contamination (V), wherein the reliability value (R) is specific to the tissue characteristic (G) that is to be determined by the evaluation device (22).

7. The tissue analysis device according to claim 1, wherein the assignment device (23) comprises a model regarding the relation between the reliability value (R) with which a specific tissue characteristic (G) is recognized and the contamination (V).

8. The tissue analysis device according to claim 7, wherein the model is a data collection.

9. The tissue analysis device according to claim 1, wherein the assignment device (23) comprises multiple transmission curve models (26) that are characteristic for different contaminations (V).

10. The tissue analysis device according to claim 9, wherein the assignment device (23) selects a matching transmission curve model (26) of the multiple transmission curve models (26) based on a spectrum (28) obtained by the spectrometer (20).

11. The tissue analysis device according to claim 9, wherein the assignment device (23) is configured to assign a reliability value (R) to each transmission curve model (26) of the multiple transmission curve models (26) for each tissue characteristic (G).

12. The tissue analysis device according to claim 1, further comprising a test device (30) for determination of the contamination (V), the test device (30) including a light source (31) that emits multi-spectral light.

13. The tissue analysis device according to claim 12, wherein the test device (30) is configured to determine the contamination (V) based on multiple light wavelengths.

14. A method for tissue analysis during a surgical intervention, the method comprising:
receiving light by a light receiving device (16), wherein the light is created due to the influence of an electrical spark (12) or plasma on tissue (11);
determining light intensities (I) at different light wavelengths (λ) from the received light;
determining data (D) characterizing at least one tissue characteristic (G) from the light intensities (I) by an evaluation device (22);
determining a contamination (V) of the light receiving device (16);
assigning a reliability value (R) to the data (D) determined by the evaluation device (22) based on the determined contamination (V);
indicating the tissue characteristic (G); and
indicating the assigned reliability value (R), if the assigned reliability value (R) is below a threshold value.

15. The method according to claim 14, further comprising defining test intervals depending on the tissue characteristic (G) to be determined.
